(19) United States
(12) Patent Application Publication
(10) Pub. No.: US 2010/0035297 A1
(43) Pub. Date: Feb. 11, 2010

(54) METHODS AND COMPOSITIONS FOR VASCULOGENIC POTENTIAL DETERMINATION

(75) Inventor: Dmitry O. Traktuev, Indianapolis, IN (US)

Correspondence Address: BARNES & THORNBURG LLP, P.O. BOX 2786, CHICAGO, IL, 60690-2786, US

(73) Assignee: INDIANA UNIVERSITY RESEARCH AND TECHNOLOGY CORPORATION, Indianapolis, IN (US)

(21) Appl. No.: 12/537,077

(22) Filed: Aug. 6, 2009

Related U.S. Application Data
(60) Provisional application No. 61/087,459, filed on Aug. 8, 2008.

(51) Int. Cl. C12Q 1/02

(52) U.S. Cl. 435/29

(57) ABSTRACT

Methods and compositions to evaluate the therapeutic vasculogenic potential of a variety of cells including endothelial cells using a monolayer of pericytic cells are disclosed. Pericytic cells or mural cells including those isolated from adipose tissue, such as for example, adipose stromal cells (ASC) or adipose-derived stromal cells (ADSCs) provide suitable conditions that stimulate endothelial cells from different origins to modulate stable vascular network organization in an in vitro experimental setting. Compositions that contain premixed ASC and EC are suitable for testing a candidate agent's angiogenic or antiangiogenic potential.

### CROSS-REFERENCE TO RELATED APPLICATIONS

This patent application claims priority to U.S. Provisional Patent Application No. 61/087,459, filed Aug. 8, 2008, the contents of which are expressly incorporated herein by reference in their entirety.

### BACKGROUND

Coronary artery disease is a major cause of morbidity and mortality, requiring bypass surgery or angioplasty in several thousands of every year. While some of these patients form collateral vessels as alternative pathways for blood supply, thus ameliorating or preventing significant ischemic myocardial damage, many do not form the vascular networks to adequately compensate for the loss of the original blood supply. Therefore, many patients require development of compositions and methods which provide additional angiogenesis. Such approaches are broadly referred to as “therapeutic angiogenesis”, and encompass both angiogenesis (capillary sprouting) and arteriogenesis (the maturation and enlargement of existing vessels).

Tissue engineering is an evolving new approach for creating replacement tissue to repair diseased tissue, including for example ischemic heart tissue and blood vessels. One tissue engineering strategy that is more common is to seed the appropriate cells on a biodegradable scaffold engineered with the desired mechanical properties, followed by stimulation of cell growth and differentiation in vitro, such that, upon implantation in vivo, the bioengineered scaffold undergoes remodeling and maturation into a functional tissue. Examples of this approach include blood vessels and other cardiovascular components, where autologous vascular cells have been used for this purpose without immune rejection.

An emerging therapeutic approach is the use of stem and progenitor cell transplantation to improve angiogenesis. For clinical applications, the presence of endothelial progenitor cells (EPCs) in circulation represents a promising opportunity to noninvasively obtain endothelial population for vasculogenesis. Endothelial progenitor cells (EPCs) are cells present in bone marrow, in peripheral blood or in the walls of the vessels. Creation of microvascular networks have been shown in vitro using biodegradable scaffolds seeded with EPCs that had been isolated from human umbilical cord blood and expanded in vitro as mature endothelial cells (ECs). In addition, human blood-derived EPCs constitute a robust source of ECs with the potential to form functional capillary networks in vivo.

Animal studies using hind limb ischemia or myocardial ischemia models in immune deficient rodents have demonstrated that transplantation of peripheral blood derived EPCs can result in increased angiogenesis. EPCs derived from bone marrow, unpurified bone marrow mononuclear cells, and HSCs have also been shown to enhance angiogenesis or show endothelial differentiation in vivo in a variety of animal models of ischemia.

Over the past decade, interest has grown in identifying and characterizing progenitor cells with vasculogenic potential isolated from a range of types of adult tissues, such as subtypes of endothelial progenitor cells, bone marrow-derived mesenchymal cells, and satellite cells from skeletal muscles. Experimental models and clinical trials have shown that these cells stimulate angiogenesis in ischemic tissues and may preserve or rescue cardiac and brain function in the context of ischemic insult.

ASC population contains cells that are able to differentiate in vitro into multiple mesenchymal cell types such as adipocytes, chondrocytes, osteoblasts as well as hepatocytes, neuronal cells, endothelial cells (ECs), and cardiomyocytes. In addition, locally or systemically injected ASCs, whether freshly isolated or expanded, stimulate angiogenesis and mediate recovery of muscle tissues following ischemic insult.

Despite advances in this field, tissue engineering still faces important constraints. There are no tissue engineering models to evaluate vasculogenic potential of cells prior to their delivery into an injured site in a patient. Strategies such as embedding angiogenic factors into the scaffold to promote ingrowth of microvessels, fabrication technologies to create polymers containing vessel-like networks, and prevascularization of matrices prior to cell seeding have been proposed. Models presently available that include one or more components of the extracellular matrix (ECM) do not adequately simulate the in vivo conditions, such as paracrine interaction and direct intercellular interaction among various cell types. Consequently, tissues implanted with even a significant cell volume suffer from proper vascularization and are not therapeutically effective.

By providing a suitable cellular background or supporting layer, the inherent vasculogenic ability of a variety of cells including endothelial cells (ECs) can be better assessed. The present disclosure relates to assessing the in vitro vasculogenic potential of a variety of cells in a setting that more likely resembles in vivo vasculature.

### SUMMARY

Methods and compositions to evaluate the therapeutic vasculogenic potential of a variety of cells including endothelial cells using a monolayer of pericytic cells are disclosed. Pericytic cells or mural cells including those isolated from adipose tissue, such as for example, adipose stromal cells (ASC) or adipose-derived stromal cells (ADSCs) provide suitable conditions that stimulate endothelial cells from different origins to modulate stable vascular network organization in an in vitro experimental setting.

Use of adipose stromal cells as an underneath monolayer of pericytes to support endothelial cells (ECs) in formation of vascular network in vitro is a consistent and a highly reproducible system used to evaluate therapeutic vasculogenic potential of ECs.

Personalized therapeutics involving delivery of cultured endothelial cells including endothelial progenitor cells (EPCs) and endothelial colony forming cells (ECFCs) on a ASCs are disclosed.

Screening methods for angiogenic and anti-angiogenic compounds that involve the use of in vitro vasculature formed ECs cultured on ASCs are also disclosed.

A method of evaluating vasculogenic potential of endothelial cells, the method includes:
(a) culturing the endothelial cells on a layer comprising a plurality of adipose stromal cells (ASC); and
(b) evaluating the vasculogenic potential by analyzing the formation of a vascular network.

In an embodiment, adipose stromal cells are pericytes. In an embodiment, the adipose stromal cells are located in the perivascular position in vivo. In an embodiment, the adipose stromal cells are mural cells. In an embodiment, the adipose stromal cells are adherent.

In an embodiment, the adipose stromal cells are freshly isolated and CD34⁺. In an embodiment, the ASCs are passaged about 1-10 or 1-20 times. In an embodiment, the adipose stromal cells are CD45⁻. In an embodiment, the adipose stromal cells are CD31^{-.}

In an embodiment, a majority of the adherent ASCs express a marker selected from the group that includes CD10, CD13, and CD90. In an embodiment a majority of the ASCs are CD45−/CD31−CD144− cells isolated from SVF.

In an embodiment, the pericytes express a marker selected from the group that includes chondroitin sulfate proteoglycan (NG2), CD140a, and CD140b (PDGF receptor- and -β, respectively).

In an embodiment, the endothelial cells are selected from the group that includes mature endothelial cells, endothelial progenitor cells (EPC) and endothelial colony forming cells (ECFC). In an embodiment, the endothelial cells are CD31⁺. In an embodiment, the endothelial cells are CD144⁺.

In an embodiment, the adipose stromal cells are grown as a monolayer. In an embodiment, the adipose stromal cells are seeded at an initial density of about 3,500-240,000 cells/cm².

In an embodiment, the endothelial cells are seeded at an initial density of about 625-25,000 cells/cm².

In an embodiment, the adipose stromal cells are cultured for about 3-24 hours prior to adding the endothelial cells. In an embodiment, the adipose stromal cells and the endothelial cells are premixed.

In an embodiment, the endothelial cells are generally homogenous. In an embodiment, the adipose stromal cells are generally homogenous. In an embodiment, the adipose stromal cells are substantially pure.

In an embodiment, the endothelial cells are derived from a source selected from the group that includes placenta, peripheral blood, cord blood, umbilical vein, cardiac microvascular cells, adipose tissue, saphenius vein, HUVEC, HmVEC, and ECFC.

In an embodiment, the adipose stromal cells form a mural angiogenic layer. In an embodiment, the mural angiogenic layer further comprises of a plurality of cells selected from the group that includes smooth muscle cells and fibroblasts.

In an embodiment, the endothelial cells are cultured on a layer substantially free of exogenously introduced one or more components of the extracellular matrix (ECM).

A method of screening a candidate angiogenic or anti-angiogenic compound/agent includes:
(a) culturing or growing adipose stromal cells and endothelial cells capable of forming a vascular network in vitro;
(b) contacting adipose stromal cells and endothelial cells with a candidate compound/agent; and
(c) determining that the candidate compound/agent is angiogenic or anti-angiogenic if the candidate compound/agent augments angiogenesis or inhibits angiogenesis by the comparing the vascular network formation to a control sample without the candidate compound/agent.

In an embodiment, the agent is a small molecule having for example less than 1000 or 500 Da. In an embodiment, the agent is a peptide or a nucleic acid. Examples of suitable nucleic acids include for example siRNA of a target gene that is involved in angiogenesis.

A method of screening a candidate angiogenic or anti-angiogenic compound, the method includes:
(a) generating an in vitro vascular network comprising culturing endothelial cells on a layer comprising adipose stromal cells; and
(b) contacting the vascular network to determine if the candidate compound augments angiogenesis or inhibits angiogenesis by the comparing the vascular network formation to a control sample without the candidate compound.

A method of generating an adequate amount of endothelial cells for in vivo vasculogenesis or angiogenesis includes:
(a) culturing the endothelial cells (ECs) on a layer comprising a plurality of adipose stromal cells (ASC); and
(b) harvesting the cultured endothelial cells.

A method of generating an adequate amount of endothelial cells for in vivo vasculogenesis or angiogenesis, the method includes:
(a) culturing the endothelial cells (ECs) on a layer comprising a plurality of adipose stromal cells (ASC);
(b) harvesting the cultured endothelial cells; and
(c) administering the cultured endothelial cells to a site in vivo.

In an embodiment, the site is an ischemic site. In an embodiment, the site is selected from the group that includes heart, brain, and skeletal muscle. In an embodiment, the injury is selected from the group that includes myocardial infarction, brain insult and peripheral vascular diseases. In an embodiment, the endothelial cells are autologous.

An angiogenic or vasculogenic composition includes a therapeutically effective amount of endothelial cells cultured on a layer comprising a plurality of adipose stromal cells (ASC). In an embodiment, the composition is substantially free of an exogenously introduced extracellular matrix component. In an embodiment, the endothelial cells include endothelial progenitor cells (EPCs). In an embodiment, the composition includes endothelial colony forming cells (ECFCs). In an embodiment, the composition includes mature endothelial cells from different origins. In an embodiment, the composition is therapeutic.

An angiogenic or vasculogenic composition includes an effective amount of endothelial cells and adipose stromal cells (ASC), wherein the adipose stromal cells and endothelial cells are mixed at a ratio of about 5:1 to about 100:1. In an embodiment, the composition is substantially free of exogenously added extracellular matrix component. In an embodiment, the composition containing the endothelial cells includes endothelial progenitor cells (EPCs) or endothelial colony forming cells (ECFCs).

An assay kit includes an effective amount of endothelial cells and adipose stromal cells (ASC), wherein the adipose stromal cells and endothelial cells (EC) are mixed at a ratio of about 5:1 to about 100:1. In an embodiment, the ration is of about 2:1 to 48:1 (ASC:EC). In an embodiment, the cells are frozen in a vial and the vial is maintained at a temperature of not higher than −80° C. In an embodiment, the cells are metabolically active (living). In an embodiment, the ASC range from about 200,000-5,000,000 cells and the endothelial cells are about 200,000-2,000,000 cells. In an embodiment, the kit further includes in separate containers, appropriate media for co-culturing ASC and EC. These media include for example ASC-preserving media and EC-preserving media. In an embodiment, the kit further includes a reagent for Platelet Endothelial Cellular Adhesion Molecule (PECAM-1/CD31) Immunoassay. Other reagents for visualization include for example, reagents for alpha-smooth muscle actin immunoassay.

An assay kit includes for example, a growth container containing a living culture of human ASC and human endothelial cells in co-culture maintaining medium; said living culture arising from the culture of an initial mixture of human ASC and human endothelial cells in a ratio of about 2:1 to 48:1 (ASC:EC) grown for a period of about 24 hours from the initial mixing of said ASC and endothelial cells and then suitably packaged.

An assay kit comprising includes for example, a growth container, a co-culture maintaining medium, a suspension of human endothelial cells, and a suspension of ASC:EC in a ratio about 2:1 to 48:1.

A kit as disclosed herein additionally includes for example, fixative for assisting in subsequent visualization, blocking buffer, washing buffer, reagents and antibodies for visualization.

### BRIEF DESCRIPTION OF THE DRAWINGS

FIG. 1 shows analysis of vascular network formation of endothelial cells (EC) grown on a monolayer of adipose stromal cells (ASC). Adipose stromal cells were plated on the cell culture treated plastic in density 50,000-240,000 cells/cm² and let adhere and spread in EBM-2/5% FBS media for 3 hours and 6 days. Then, a density of about 5,000-25,000 cells/cm² of endothelial cells in EBM-2/5% FBS media was added on the top of ASC monolayer. Endothelial cells for this assay were isolated several sources: Adipose tissue derived EC, placenta derived EC, saphenuis vein EC, HUVEC, HmVEC, ECFC). Media was exchanged at day 3. Analysis of network formation was performed under a phase contract microscope at day 6.

FIG. 2 shows fluorescent images of endothelial cells on ASC and vascular network formation. Titration of endothelial cell number on ASC monolayer was performed as described herein. Adipose stromal cells were plated in density 65,000/cm2 and let adhere and spread in EBM-2/5% FBS media for 3 hours, followed by adding on the top of ASC monolayer 625-10,000/cm² of blood derived endothelial cells in EBM-2/5% FBS media. Media was exchanged at day 3. At day 6, wells with the co-cultures were fixed with methanol, stained for human CD31 antigen and the antigen-antibody complex was revealed by incubating the wells with secondary antibodies conjugated to Alexa 488. Analysis of network formation was performed under fluorescent microscope at 4× magnification.

FIG. 3 shows quantitative analysis of titration of endothelial cell number on ASC monolayer. Digital pictures were processed with MetaMorph software (Molecular Devices, Sunnyvale, Calif.). The data that was extracted included: (1) total tube length; (2) mean tube length; (3) total tube area; (4) mean tube area, (5) tube % area covered; (6) average tube thickness; (7) segments; branch points. The graphs shown in FIG. 3 are for total tube length and branch points.

FIG. 4 demonstrates that ASC have a higher ability to support vascular network formation by ECs in an in vitro model when compared to smooth muscle cells and fibroblast in their ability to support vasculature. Human smooth muscle cells from carotid artery and aorta, human ASCs and human dermal fibroblasts were plated in a density of about 65,000 cells/cm² and let adhere and spread in EBM-2/5% FBS media for 3 hours, followed by the addition of a density of about 5,000 cells/cm² of blood derived endothelial cells (ECs) in EBM-2/5% FBS media on top of the ASC monolayer. Media was exchanged at day 3. At day 6, wells with the co-cultures were fixed with methanol, stained for human CD31 antigen and were analyzed under a fluorescent microscope at 4× magnification. The graphs shown on FIG. 4 are for total tube length and branch points.

FIG. 5 demonstrates that ASC support vascular network formation by EC isolated from different sources: adipose tissue, placenta, saphenius vein, umbilical vein, cardiac microvascular ECs and blood derived ECs. ASCs were plated in a density of about 65,000 cells/cm² and let adhere and spread in EBM-2/5% FBS media for 3 hours, followed by the addition of a density of about 5,000 cells/cm² of endothelial cells (ECs) from different sources in EBM-2/5% FBS media. Media was exchanged at day 3. At day 6, wells with the co-cultures were fixed with methanol, stained for human CD31 antigen and were analyzed under a fluorescent microscope at 4× magnification. The graphs shown on FIG. 5 are for total tube length and branch points.

FIG. 6 demonstrates that vascular network formation by EC on ASC monolayer can be described by presence in the culture media anti-angioogenic factors, such as anti-angiogenic compound Gleevec™ or anti-VEGF antibodies. ASCs were plated in a density of about 65,000 cells/cm² and let adhere and spread in EBM-2/5% FBS media for 3 hours, followed by adding on the top of ASC monolayer 5,000 cells/cm² of EC in EBM-2/5% FBS alone (control) or in EBM-2/5% FBS supplemented with Gleevec™ (1 mM), anti VEGF antibodies (2 μg/ml) or isotype control antibodies (2 μg/ml).

Medium with the same supplements were exchange at day 3. At day 6, wells with the co-cultures were fixed with methanol, stained for human CD31 antigen; 5-9 pictures/well at 4× were taken under fluorescent microscope and were analyzed for total tube length and branch points.

FIG. 7 shows that vascular networks formed by combination of ASC and EC are stable for at least 21 days. Human ASCs were plated in a density of about 60,000 cells/cm2 and let adhere and spread in EBM-2/5% FBS media for 3 hours, followed by the addition of a density of about 5,000 and 10,000 cells/cm² of blood derived endothelial cells (ECs) in EBM-2/5% FBS media on top of the ASC monolayer. Media was changed every 3-4 days. One third of the wells with co-cultures of each kind were fixed with methanol at day 8, day 14 and day 21, followed by staining for human CD31 antigen and analysis under a fluorescent microscope at 4× magnification for total tube length.

FIG. 8 shows that EC plated simultaneous with ASC have demonstrated higher degree of network development compare with serial (layering) plating. ECs are able to establish vascular network to a higher degree in an in vitro model when are mixed with ASC prior to plating into the wells compare with EC that were plated on the established monolayer of ASCs. In the half of the wells (labeled on the graph as “Layer”) human ASCs were plated in a density of about 60,000 cells/cm2 and let adhere and spread in EBM-2/5% FBS media for 3 hours, followed by the addition of a density of about 5,000 cells/cm2 of blood derived endothelial cells (ECs) in EBM-2/5% FBS media on top of the ASC monolayer. In the other half of the wells (labeled on the graph as “mix”) EC (about 5,000 cells/cm²) and ASC (about 60,000 cells/cm2) were mixed/combine in the tube in EBM-2/5% FBS media prior to plating into the wells. Media was exchanged at day 3. At day 6, wells with the co-cultures were fixed with methanol, stained for human CD31 antigen and were analyzed under a fluorescent microscope at 4× magnification for total tube length.

FIG. 9 shows quantitative analysis of titration of ASC number used for monolayer formation. Human ASCs were plated in a density of about 30-240,000 cells/cm2 and let adhere and spread in EBM-2/5% FBS media for 3 hours, followed by the addition of a density of about 5,000 cells/cm² of blood derived endothelial cells (ECs) in EBM-2/5% FBS media on top of the ASC monolayer. Media was exchanged every 3 days. At day 6, wells with the co-cultures were fixed with methanol, stained for human CD31 antigen and the antigen-antibody complex was revealed by incubating the wells with secondary antibodies conjugated to Alexa 488. Analysis of network formation (total tube length) was performed under fluorescent microscope at 4× magnification. No significant different in the degree of vascular network development was observed as a results of number of ASC used to form the monolayer (in the range of numbers used).

FIG. 10 shows (A) Representative images of ASC monoculture (left) and EC-ASC co-cultures (middle and right) that were probed for αSMA (red) at day 6 post plating. Nuclei revealed by DAPI staining. B, Analysis of CD31 antigen expression on EC cultures alone or together with ASCs during the 6 day-period post plating (n=10). C, Analysis of ECs and ASCs persistence in co-cultures during 6 day period. (n=12, **p≦0.01, ***p≦0.001).

### DETAILED DESCRIPTION

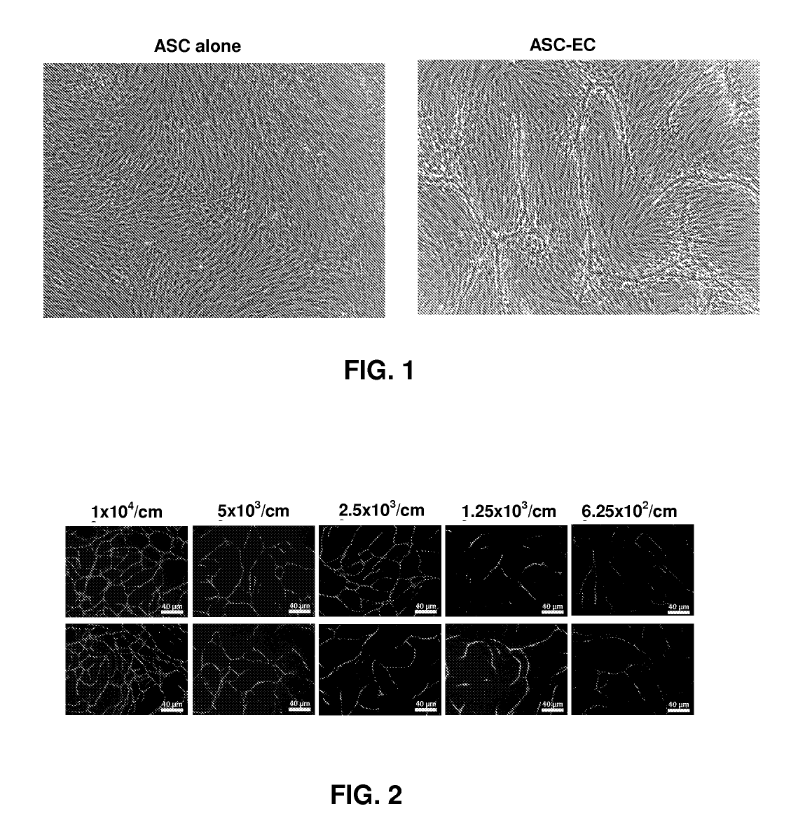

As demonstrated herein, vascular network formation of endothelial cells (EC) grown on a monolayer of adipose stromal cells (ASC) is shown in FIG. 1. Adipose stromal cells were plated on the cell culture treated plastic in density 50,000-65,000 cells/cm² and let adhere and spread in EBM-2/5% FBS media for about 3-24 hours. Then, a density of about 10,000 cells/cm² of endothelial cells in EBM-2/5% FBS media was added on the top of ASC monolayer. Endothelial cells for were isolated several sources: Adipose tissue derived EC, placenta derived EC, saphenuis vein EC, HUVEC, HmVEC, ECFC. Fluorescent images of endothelial cells on ASC and vascular network formation are shown in FIG. 2. Titration of endothelial cell number on ASC monolayer was performed as described herein. Quantitative analysis of titration of endothelial cell number on ASC monolayer is shown in FIG. 3. The data that was collected included: (1) total tube length; (2) mean tube length; (3) total tube area; (4) mean tube area, (5) tube % area covered; (6) average tube thickness; (7) segments; branch points. The graphs shown in FIG. 3 are for total tube length and branch points.

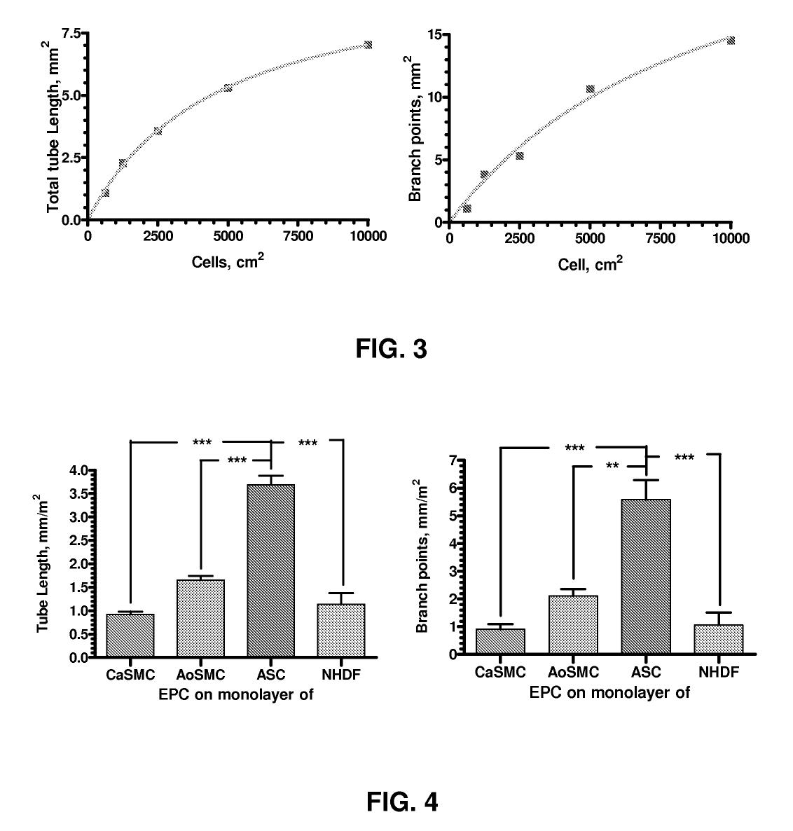

ASC have a higher ability to support vascular network formation by ECs in an in vitro model when compared to smooth muscle cells and fibroblast in their ability to support vasculature (FIG. 4). Human smooth muscle cells from carotid artery and aorta, human ASCs and human dermal fibroblasts were plated in a density of about 65,000 cells/cm² and let adhere and spread in EBM-2/5% FBS media for 3 hours, followed by the addition of a density of about 5,000 cells/cm² of blood derived endothelial cells (ECs) in EBM-2/5% FBS media on top of the ASC monolayer. At day 6, wells with the co-cultures were fixed with methanol, stained for human CD31 antigen and were analyzed under a fluorescent microscope at 4× magnification. The graphs shown on FIG. 4 are for total tube length and branch points.

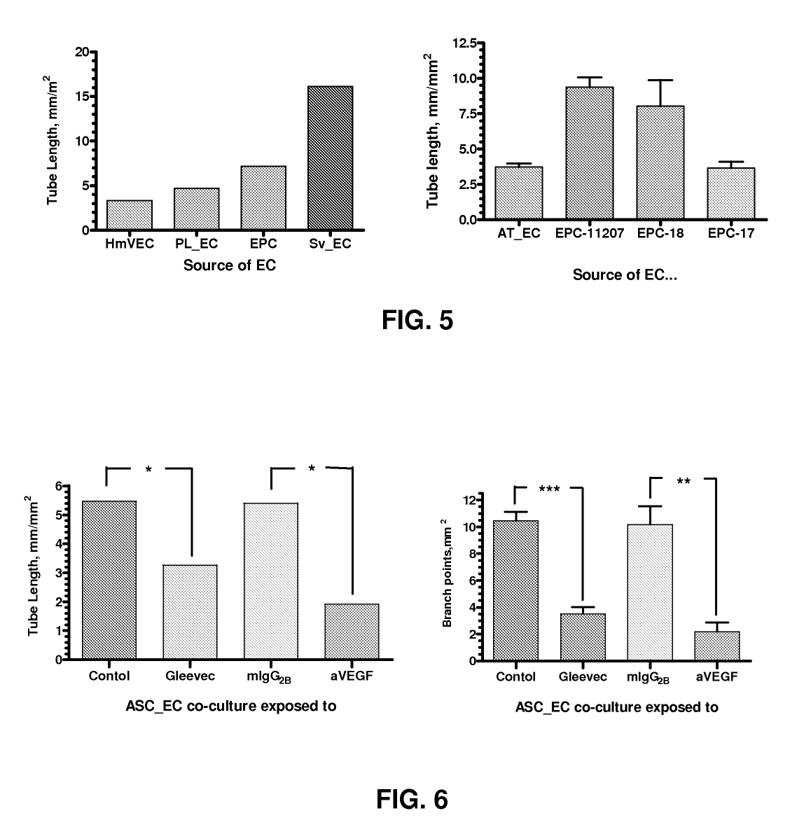

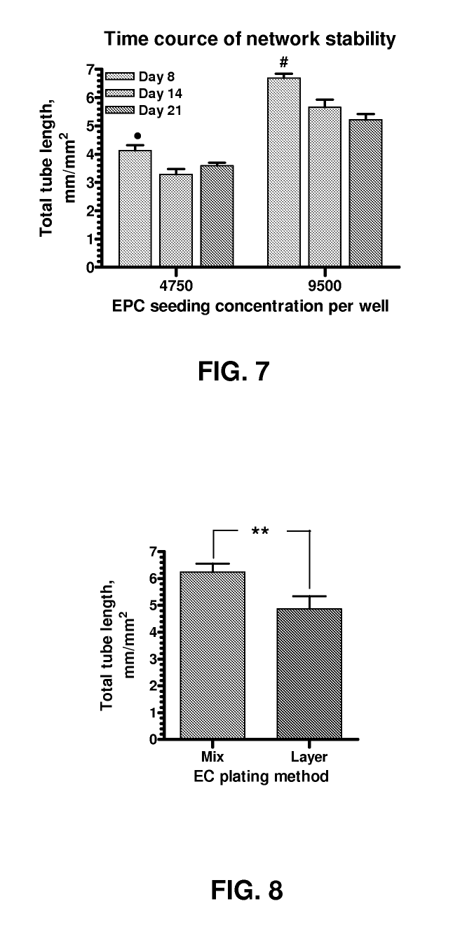

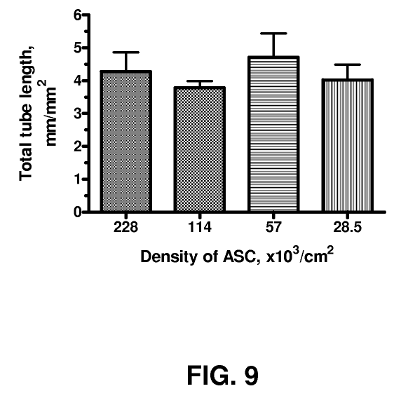

As shown in FIG. 5, ASCs support vascular network formation by EC isolated from a variety of sources including adipose tissue, placenta, saphenius vein, umbilical vein, cardiac microvascular ECs and blood derived ECs. The graphs shown on FIG. 5 are for total tube length and branch points.

As shown in FIG. 6, vascular network formation by EC on ASC monolayer is elucidated by anti-angiogenic compound Gleevec™ or anti-VEGF antibodies.

Stem or progenitor cells in many tissues are deployed on blood vessels. Pericytes isolated from different tissues are pluripotent. Mesenchymal stem cells (MSCs) are found in perivascular sites in many tissues: in bone marrow, where they interact with both sinusoidal ECs and hematopoietic precursors; in central nervous system; in dental pulp; and in others. Multipotent ASCs also have a perivascular location and express pericytic markers. Analysis of all these findings demonstrates that in adult tissues, the perivascular compartment represents a niche for multipotent cells. The interaction of EC with these multipotent cells, including ASCs, shows that endothelium modulates their function within this niche.

ASCs express some of the tested pericyte markers CD140a, CD140b, NG2, and -smooth muscle actin occasionally express CD146, also a pericyte marker. ASCs may be described as a cell with pericytic properties. Pericytes from a range of tissues differ in antigen expression because of unique local environments. Pericytic identity may be established by physiological properties and function in addition to designation by surface markers alone.

The data provided herein demonstrate a model for a sequence of events leading to initiation, development and stabilization of vascular structures formation in vitro. EC contact with ASC leads to modulation of growth factor production by both cell types, facilitating EC survival. Concurrent accumulation of ECM produced by ASCs as well as ECs induces the ECs to assemble into cord structures which correlate with increased expression of PECAM-1 on the EC surface; this has been described as a key sign of initial vessel maturation). The formation of cord structures is accompanied by induction of an EC secretory program that in turn stimulates ASC migration and further accumulation into regions near the EC cords. The ASCs become incorporated into the vessel “wall”, transforming from αSMAdim into αSMAbright expressing cells, likely developing initial contractility of the developing vessels. This sequence of events may lead to multilayered mature vessel formation, which will correlate with gradual down-regulation of angiogenic factors and matrix protein production.

This in vitro demonstration that plating ECs and ASCs together induces joint organization into vascular cord structures, provides deeper understanding of the observation that mixtures of EC with ASC were able to establish multilayered functional vessels in mouse tissue (ear pinnae) even without additional exogenously added ECM proteins support. These observations also strongly support a primary concept that local injection of ECs-ASCs mixtures can significantly increase blood supply in under-perfused tissues by promoting assembly of ECs into multilayer vessels and establishing connections with the host vasculature.

The present model is free from exogenously added ECM proteins as well as additional cytokines and growth factors (only exposed to the 5% serum). These features of the proposed model system make the analysis of the communications (direct physical contact and paracrine crosstalk) between complementary vasculogenic cell types less susceptible to the confounding effects of the introduction of exogenous factors to the system.

The observations that certain types of adult progenitor cells can be efficiently expanded in vitro without losing angiogenic properties has raised the enthusiasm for the utility of autologous cardiovascular cell therapies. However, the absence of reliable methods to validate the therapeutic potentials of the isolated/expanded cells prior to implantation creates uncertainty in predicting the efficacy of these therapies. For example, it is known that physiological and pathological factors such as age, diabetes, and hyperglycemia significantly decrease the therapeutic potential of the cells. However, there are many other undefined, subject-specific factors that can significantly compromise cell therapeutic potentials. Pre-testing for cell potency in vitro using the angiogenic models disclosed herein prior to injection, provides a priori knowledge about the efficacy of a cell treatment. By using well-defined “gold standard” endothelial cells and well-defined “mural cells”, e.g., a population of ASCs as described in this report, an assay is readily conducted to screen the vasculogenic/angiogenic potential of each progenitor cells prior to implantation.

The simplicity of the model setup and its duration (about 3-6 days), as well as data acquisition potentially automated computer-assisted analysis renders this model useful for testing different pro- and anti-angiogenic compounds.

In an embodiment, a highly defined ASC population (CD34+/140a+/140b+/31−/45−/117−/144−) is a subset of adipose-derived cells, which in quiescent adipose tissue, possesses a majority of pericytic properties, while harboring the ability to enter into multiple other distinct lineages.

In an embodiment, the vasculogenic potential of ECs is evaluated in the absence of a significant amount of an exogenously introduced one or more components of the extracellular matrix (ECM) as a support for vascular network formation.

In an embodiment, adipose stromal cells are plated on a cell culture treated plastic substrate at an initial density of about 3,500-50,000 cells/cm² or about 30,000-65,000 cells/cm² or about 50,000-65,000 cells/cm² or about 40,000-75,000 cells/cm² or greater than about 50,000 cells/cm² or about 50,000-250,000 cells/cm² or higher.

In an embodiment, ASCs are suitable for monolayer formation for vasculogenic assays after fresh isolation. In an embodiment, ASCs up to passage 10 or higher is suitable. In an embodiment, ASCs are passaged 1-10 times or about 5 times or 1-7 times prior to being assayed.

In an embodiment endothelial cells (ECs) are plated at an initial density of about 650-25,000 cells/cm² or 500-5000 cells/cm² or 5000-10000 cells/cm² or 500-10000 cells/cm² or greater than about 500 cells/cm² or about 10,000-50,000 cells/cm² or about 5000 cells/cm².

In an embodiment, the ratio of ASC: EC may range from about 2:1, 5:1, 6:1, 10:1, 12:1, 15:1, 20:1, 25:1, 30:1, 35:1, 40:1, 50:1, 100:1, and 200:1 as long as vascular network formation is not significantly affected.

In an embodiment, the ASC and EC are pre-mixed prior to culture. In another embodiment, the ASC and EC are simultaneously mixed and plated or cultured for assaying vasculogenic potential or for screening antiangiogenic compounds. Pre-mixed or co-mixed cells of ASC and EC are frozen for example in liquid nitrogen involving appropriate stabilizers such as, for example, glycerol and maintained at −80° C.

A pericyte, may also be known as a Rouget cell or mural cell that is generally associated with the walls of blood vessels, especially small blood vessels. As a relatively undifferentiated cell, it serves to support these vessels, but it can differentiate into a fibroblast, smooth muscle cell, or macrophage as well if required. They are important in blood brain barrier stability as well as angiogenesis.

As used herein, the term “adipose tissue” refers to a tissue containing multiple cell types including adipocytes and microvascular cells. Adipose tissue includes stem cells and endothelial precursor cells. Accordingly, adipose tissue refers to fat including the connective tissue that stores the fat. By “adipose” is meant any fat tissue. The terms “adipose” and “adipose tissue” are used interchangeably herein. The adipose tissue may be brown or white adipose tissue, derived from subcutaneous, omental/visceral, mammary, gonadal, or other adipose tissue site. Preferably, the adipose is subcutaneous white adipose tissue. Such cells may comprise a primary cell culture or an immortalized cell line. The adipose tissue may be from any organism having fat tissue. Preferably, the adipose tissue is mammalian, most preferably, the adipose tissue is human. A convenient source of adipose tissue is from liposuction surgery, lipoectomy or procedures such as reduction mammoplasty, however, the source of adipose tissue or the method of isolation of adipose tissue does not limit the practice of the vasculogenesis assays disclosed herein.

The term “adipose stromal cells” or ASC refers to a population of cells obtained from adipose stromal fraction derived from adipose tissue. These include adipose derived stem cells. For example, the ASC are multipotential mesenchymal progenitor cells that may be induced to undergo adipogenic differentiation, and may have functional and phenotypic overlap with pericytes lining microvessels in adipose tissues. The cells can be fresh, or in culture. Adipose stromal cells contain pluripotent cells, which have the ability to differentiate into cell types including but not limited to adipocytes, cardiomyocytes, endothelial cells, hematopoietic cells, hepatic cells, chondrocytes, osteoblasts, neuronal cells, and myotubes. In an embodiment, adipose stem cells from mouse for example, may include cells within the adipose stromal fraction which exhibit a stem cell phenotype, such as for example CD45−/Sca-1+/c-kit- or or CD45−/CD34+/c-kit-.

In an embodiment, adipose stromal cells are generally homogenous. In an embodiment, the adipose stromal cells are substantially homogenous, except for the variations exhibited by cells that are cultured, i.e., culture artifacts. For example, in a population of cells derived from adipose stroma, the ASC population may be greater than about 75% or 80% or 85% or 90% or 95% or 99% homogenous in at least one of surface marker expression or phenotypic or functional characteristics. In an embodiment, the isolated ASCs may be substantially pure and substantially free of non ASCs. For example, the isolated and purified population of ASCs may be greater than about 75% or 80% or 85% or 90% or 95% or 99% pure.

The term “endothelial cells” (EC) refer to a cell of endothelial origin including mature or semi-mature or partially-mature population of endothelial cells isolated from different tissues and organs, endothelial progenitor cells (EPC), endothelial colony forming cells (ECFC), circulating and fixed endothelial cells, endothelial cells isolated from cord blood, peripheral blood, adult blood, blood vessels, clonally propagated endothelial and endothelial progenitor cells, low and high proliferating potential ECFC and those that are differentiated from a progenitor cell such as a from a stem cell.

The term “monolayer” as used herein can refer to cells that are attached to a solid support while proliferating in suitable culture conditions. A small portion of cells proliferating in a monolayer under suitable growth conditions may be attached to cells in the monolayer but not to the solid support. Preferably less than 15% of these cells are not attached to the solid support, more preferably less than 10% of these cells are not attached to the solid support, and most preferably less than 5% of these cells are not attached to the solid support.

The term “plated” or “plating” or “seeding” as used herein in reference to cells can refer to establishing cell cultures in vitro. For example, cells can be diluted in cell culture media and then added to a cell culture plate, dish, or flask. Cell culture plates are commonly known to a person of ordinary skill in the art. Cells may be plated at a variety of concentrations and/or cell densities.

The term “cell plating” can also extend to the term “cell passaging.” Cells of the invention can be passaged using cell culture techniques well known to those skilled in the art. The term “cell passaging” can refer to a technique that involves the steps of (1) releasing cells from a solid support or substrate and disassociation of these cells, and (2) diluting the cells in media suitable for further cell proliferation. Cell passaging may also refer to removing a portion of liquid medium containing cultured cells and adding liquid medium to the original culture vessel to dilute the cells and allow further cell proliferation. In addition, cells may also be added to a new culture vessel which has been supplemented with medium suitable for further cell proliferation.

The term “proliferation” as used herein in reference to cells can refer to a group of cells that can increase in number over a period of time.

The term “feeder cells” as used herein can refer to cells that are maintained in culture and are co-cultured with target cells. Target cells can be cultured cells, stem cells, endothelial cells, endothelial progenitor cells for example. Feeder cells can provide, for example, peptides, polypeptides, electrical signals, organic molecules (e.g., steroids), nucleic acid molecules, growth factors (e.g., bFGF), other factors (e.g., cytokines such as LIF and steel factor), and metabolic nutrients to target cells. Certain cells may not require feeder cells for healthy growth. Feeder cells preferably grow in a mono-layer.

As used herein, the phrase “cardiovascular condition, disease or disorder” is intended to include all disorders characterized by insufficient, undesired or abnormal cardiac function, e.g., ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death. As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results from an occlusive (e.g., thrombotic or embolic) event in the coronary circulation and produces an environment in which the myocardial metabolic demands exceed the supply of oxygen to the myocardial tissue.

The term “peripheral vascular disease” (PVD) or “peripheral arterial disease” (PAD) or “peripheral artery occlusive disease” (PAOD), refer to diseases caused by an obstruction of peripheral arteries (e.g., outside of heart and brain), which can result from atherosclerosis, inflammatory processes leading to stenosis, or an embolism or thrombus formation. It causes either acute or chronic ischemia (lack of blood supply), for example, typically of the legs. It also refers to arteriosclerosis of the extremities, a disease of the blood vessels that leads to narrowing and hardening of the arteries that supply a variety of organs including lungs, kidneys, legs and feet.

The term “brain insult” or “brain injury” refers to hypoxic injury or ischemic brain injury to the brain (e.g, a stroke that occurs when the blood supply to any part of the brain is interrupted, resulting in tissue death and loss of brain function), which can occur during cerebral ischemia or high-altitude edema.

As used herein, the term “vasculogenesis” refers to the de novo formation of new blood vessels. The term “vasculogenic potential” refers to the ability of endothelial cells including progenitor and colony forming cells to form blood vessels or blood vessel-like vascular network in vitro.

As used herein, the term “angiogenesis” refers to the process by which new blood vessels are generated from existing vasculature and tissue. The phrase “repair or remodeling” refers to the reformation of existing vasculature. The alleviation of tissue ischemia is dependent upon angiogenesis. The spontaneous growth of new blood vessels provides collateral circulation in and around an ischemic area, improves blood flow, and alleviates the symptoms caused by the ischemia. As used herein, the term “angiogenic factor” or “angiogenic protein” refers to any known protein capable of promoting growth of new blood vessels from existing vasculature (“angiogenesis”).

As used herein, the term “arteriogenesis” refers to the process of enhancing growth of collateral arteries and/or other arteries from pre-existing arteriolar connections. More particularly, arteriogenesis is the in situ recruitment and expansion of arteries by proliferation of endothelial and smooth muscle cells from pre-existing arteriolar connections supplying blood to ischemic tissue, tumor or site of inflammation.

The term “consisting essentially of” refers to compositions that contain ASCs and may optionally contain any other components that do not materially affect the functional attributes of ASCs e.g., vasculogenic potential of ECs. For example, a composition consisting essentially of ASCs may include other factors that may affect the vasculogenesis of ECs.

### Examples

The following examples are for illustrative purposes only and are not intended to limit the scope of the disclosure.

### Example 1

### ASCs Support Vasculogenesis by ECs

Robust practical ways to assess the therapeutic vasculogenic potential of endothelial cells prior to their delivery into patients for treatment of local ischemia are not available. Methods used to evaluate vasculogenic potential of the cells in vitro included plating the endothelial cells on the surface of the different types of extracellular matrix proteins, e.g., either pure (collagens) or their combinations (matrigel). These methods are limited in their ability to modulate the conditions present in vivo, such as paracrine and direct interaction between ECs and surrounding cells, especially with mural cells/pericytes. Moreover all previous methods were generally used to screen potential angiogenic and anti-angiogenic compounds in vitro and did not directly address the therapeutic efficacy of angiogenesis in vivo.

As shown herein (FIGS. 1-5), the monolayer of pericytic cells (e.g., particularly those isolated from adipose tissue, adipose stromal cells or ASC) are able to provide the conditions that stimulate endothelial cells from different origins to modulate stable vascular network organization in an in vitro experimental setting. As shown in FIG. 6, vascular network formation by EC on ASC monolayer is elucidated by anti-angiogenic compound Gleevec™ or anti-VEGF antibodies.

Use of adipose stromal cells (ASCs) as an underneath monolayer of pericytes to support endothelial cells in formation of vascular network in vitro is a practical and clinically relevant approach due to the relative ease with which one can obtain adipose stromal cells in a significant amount. This system enables high consistency and is capable of evaluating the vasculogenic potential of a variety of cells isolated from a variety of sources.

Cord blood derived endothelial cells (ECFC) on monolayer of ASC spontaneously assemble into vascular structures in vitro. Additionally, correlation between the amount of plated endothelial cells and the degree of network development (based on total tube length and branching) is also shown. The vascular network develops on ASC monolayer by EC isolated from multiple sources: placenta, cord blood, umbilical vein, cardiac microvascular cells, adipose tissue, saphenius vein. The mural angiogenic layer can be formed by multiple cells including smooth muscle cells, fibroblasts and ASC, however ASC monolayer has profound ability to support vascular network development.

Adipose stromal cells were plated on the cell culture treated plastic in density 50,000-65,000 cells/cm² and let adhere and spread in EBM-2/5% FBS media for 3-24 hours, followed by plating on the top of ASC monolayer 650-10,000 cells/cm² of endothelial cells from different origins (Adipose tissue derived EC, placenta derived EC, saphenuis vein EC, HUVEC, HmVEC, ECFC) in EBM-2/5% FBS media. Analysis of network formation evaluated under microscope revealed significant vascular network formation by day 3 and continue to remodel the next 3 days. By day 6 most of endothelial cells were recruited into the vascular network. ASC in direct contact with endothelial cell network or in close proximity to ECs demonstrated upregulation of alpha smooth muscle actin expression.

### Example 2

### Defining the Subpopulation of SVF Corresponding to ASCs

Multilabel flow cytometric analysis was performed using markers that allowed differentiation among 3 main cell types: ASCs, ECs, and leukocytes. In addition, dissimilar attachment was used and growth properties to further distinguish these. Endothelial and leukocyte cells use protein matrices to promote attachment and growth in culture. A limited plating time (1 to 2 days) of SVF on uncoated tissue culture plastic resulted in selective adherence of a population that was greatly reduced in the percentage of endothelial (CD31 and CD144) and leukocytic (CD45) cells. Staining of freshly isolated cells for CD34 and CD45 revealed 3 major subpopulations: CD34−/CD45+, CD34+/CD45+, and CD34+/CD45−. The CD45+ cells (47.6±5.8% of SVF) likely include a mixture of leukocytes contained within vasculature as well as resident in the adipose tissue. Analysis of the CD34+/CD45− population revealed a significant subpopulation (20±5%) that coexpressed the EC markers CD31 and CD144. Culturing isolated cells on plastic resulted in enrichment to >85% of cells bearing the surface marker profile CD34+/CD31−/CD45−/CD144−. Accordingly, this adherent fraction of nonendothelial CD34+/CD31−/CD45−/CD144− cells broadly defines the ASC population that has been previously characterized as pluripotent. The differentiation capacity of this purified population was confirmed by the demonstration that these undergo both osteogenesis and adipogenesis at high frequency.

Isolation and Culture of Human ASCs: Human subcutaneous adipose tissue samples (N=10), obtained from lipoaspiration/liposuction procedures were digested in collagenase type I solution (Worthington Biochemical, Lakewood, N.J.) under agitation for 2 hours at 37° C. and centrifuged at 300 g for 8 minutes to separate the stromal cell fraction (pellet) from adipocytes. The pellet was resuspended in DMEM/F12 containing 10% FBS (Hyclone, Logan, Utah) filtered through 250-μm Nitex filters (Sefar America Inc, Kansas City, Mo.) and centrifuged at 300 g for 8 minutes. The cell pellet was treated with red cell lysis buffer (154 mmol/L NH₄Cl, 10 mmol/L KHCO₃, 0.1 mmol/L EDTA) for 10 minutes. The final pellet was resuspended in EBM-2/5% FBS or EGM2-MV (Cambrex, Baltimore, Md.).

Flow Cytometric Characterization of Human ASCs: Freshly isolated SVF cells, and cells cultured for 2 days on culture plastic, were analyzed for surface marker expression using a Calibur flow cytometer analyzer and Cell QuestPro software (Becton Dickinson Immunocytometry Systems). Day 2 cells were harvested with 2 mmol/L EDTA/PBS. All of the following steps were performed on ice. Cell pellets were incubated for 20 minutes with primary antibodies or matching isotype controls (5 μg/mL). The primary antibodies used were CD10-phycoerythrin (PE), CD13-PE, CD31-PE, CD45-fluorescein isothiocyanate, CD34-allophycocyanin, CD90-PE, CD140a-PE, CD140b-PE, CD144-PE, and chondroitin sulfate proteoglycan (NG2) (Chemicon, Temecula, Calif.). To detect nonlabeled primary antibodies, samples were incubated for 20 minutes with PE-conjugated antibodies (BD, San Diego, Calif.), then washed with 2% FBS/PBS, and fixed with 2% paraformaldehyde.

Immunofluorescent Analysis of Adipose Tissue: Frozen sections of human fat tissue were simultaneously stained with rabbit anti-CD31 and mouse anti-human CD34 or with mouse anti-human CD31 and rabbit anti-CD140b antibodies.

Immunofluorescent Analysis of Isolated ASCs: Freshly isolated ASCs were stained by immunofluorescence, 3 days after plating, against caldesmon, calponin, and α-smooth muscle actin antigens.

Matrigel Assay: Human microvascular ECs (HMVECs) (passage 7) and ASCs (day 2 or passage 1) were labeled with PKH2 (green) and PKH-26 (red) (Sigma, St Louis, Mo.), respectively. Twelve-well plates were coated with 600 μL per well of growth factor-reduced Matrigel (BD). Plates were incubated at 37° C. for 2 hours. Cells (10⁵ cells/well). HMVECs and ASCs were plated separately or together at a ratio of 1:3 (7.5×10⁴ HMVECs+2.5×10⁴ ASCs) in 600 μL of DMEM/10% FBS and were cultured at 37° C. with 10% CO₂ and monitored frequently by fluorescent microscopy.

Generation of Conditioned Media: Human (h)ASCs and HMVECs were grown in EGM-2MV, in T75 flasks, until confluent, and media were changed to 10 mL of EBM-2/5% FBS. Seventy-two hours later, conditioned media (CM) was collected, centrifuged at 300 g for 5 minutes, and frozen at −80° C. Cell counts were determined in a standard manner.

Proliferation Assay: Cells were grown for 24 hours in EBM-2/5% FBS before detachment with 0.05% trypsin/EDTA and replating into 12-well plates at 10⁴ cells per well. Four hours postplating, media were changed to fresh EBM-2/5% FBS (control) or CM prepared as above. Four days later, cells were detached and counted.

Migration Assay: The bottom surfaces of Costar Transwell insert membranes, with pore diameters of 5 μm for ASCs and 8 μm for HMVECs, were coated with 50 μg/mL rat tail collagen I (BD). Cells (3×10⁴) in 0.1 mL of EBM-2/5% FBS were added into each insert, and the inserts were placed into 24-well plates, with wells containing either controls or CM. Cells were allowed to migrate for 4 hours, after which the downward aspect of the inserts were stained for transmigratory cells with Diff-Quick (Dade Behring). Cells retained on the top surface of the membranes were eliminated using a cotton swab. Insert undersides were imaged and migratory cells quantitated using ImageJ software.

Evaluation of hASC Mitogenic Response to Individual Growth Factors: Freshly isolated ASCs were adhered on cell culture plastic for 2 days before harvesting attached cells with 0.05% trypsin/EDTA. Cells were seeded at 3×10³ cells/cm² in 12-well plates and allowed to attach overnight in EBM-2/5% FBS medium, which was then replaced with EBM-2/5% FBS alone (control) or EBM-2/5% FBS supplemented with basic fibroblast growth factor (bFGF), epidermal growth factor (EGF), or platelet-derived growth factor (PDGF)-BB individually at final concentrations of 10 pg, 100 pg, 1 ng, or 10 ng each or the mixture of all of these, each at the specified concentration. Cells were cultured for 4 days, with a medium change after 48 hours, and then detached using 0.05% trypsin/EDTA, and viable cells were counted using a hemacytometer and trypan blue. Proliferation data are presented as the ratio of cells per well supplemented with growth factor to cells per well in control media.

This study demonstrates that stromal cells derived from subcutaneous adipose tissue, which have properties of preadipocytes and manifest clonal pluripotency along multiple lineage pathways serve structurally and functionally as pericytes within adipose tissue. ASSc promote vascularization and accelerate tissue perfusion in the context of ischemia.

CD34 is present on freshly isolated ASCs, occurring in the absence of CD45, which distinguishes ASCs from cells of hematopoietic lineage. The variability in reports of ASC as CD34+, as well as CD34− cells is likely a consequence of its consistent downregulation during culturing over a several-day period; passaged ASCs have been characterized as CD34low or CD34−. CD34 antigen may serve as a convenient tool for identifying the majority of freshly isolated ASCs, when used with the CD45− and CD31− phenotypes to exclude leukocytic and endothelial populations respectively.

This study also demonstrates that the marker profile of ASCs shares much in common with bone marrow-derived MSCs, as also found in analyses of gene expression. One apparent distinction is a widespread and prominent degree of CD34 expression on ASCs, contrasting with descriptions of MSCs as lacking CD34. ASC expression of CD34 is rapidly downregulated in culture, and that most isolation protocols for MSCs require extended culture periods before availability of MSCs for study. MSC in situ may indeed express the CD34 antigen but this property is lost during expansion between isolation and surface marker evaluation. Indeed, early descriptions of MSCs reported enrichment of their colony-forming activity using CD34+ selection.

### Example 3

### CD34+ Cells in Adipose Tissue are Associated with the Vasculature

Immunofluorescent analysis of human adipose tissue sections established that the majority of CD34+ cells were associated with vessels within the tissue. A significant portion of these cells also coexpressed CD31, and therefore were presumably capillary ECs. However, a separate and predominant population of CD34+ cells, which did not express CD31, was observed in proximity to ECs. The perivascular location of the CD34+/CD31− cells suggested their pericytic identity.

### Example 4

### Adipose-Derived CD34+/CD31− Cells Display Pericytic Markers In Vitro

The predominantly periendothelial location of the CD34+/CD31− cells indicated that these cells were pericytes, mural cells, which line and stabilize vascular endothelium in vivo. The adherent CD34+/CD31− population of SVF was characterized for expression of mesenchymal, pericytic, and smooth muscle cell markers. More than 95% of the adherent CD34+/CD45−/CD31− population coexpressed the mesenchymal cell markers CD10, CD13, and CD90. Analysis of surface markers used to define pericytes revealed that the majority of the ASCs expressed chondroitin sulfate proteoglycan (NG2), CD140a, and CD140b (PDGF receptor- and -β, respectively). Analysis of cytoskeletal markers performed by immunocytochemistry of freshly isolated ASCs revealed that many ASCs also expressed the smooth muscle cell antigens caldesmon, calponin, and -smooth muscle actin.

The perivascular location of CD34+/CD31− cells suggested a pericytic identity, which was confirmed by flow cytometry and further immunolocalization of these cells using a panel of markers associated with pericytes, including CD140a and CD140b (PDGF receptor- and -β), and NG2. Staining for these markers in adipose tissue confirmed a perivascular location for the ASC population. It is notable that these markers were present on most ASCs, as defined above (>95%), reflecting that pericytic cells are not a minor subset of ASCs isolated using standard methods but rather are substantially identical to these cells, at least in subcutaneous adipose tissue.

### Example 5

### ASCs Occupy a Perivascular Position in Adipose Tissue In Vivo

The spatial relationship between EC and ASCs was evaluated by colabeling adipose tissue sections with antibodies specifically decorating each cell type. The surface antigen CD140b was highly expressed by ASCs but not microvascular EC, whereas, as shown above, ASCs did not express the EC antigen CD31. Cells comprising capillary vessels in adipose tissue exhibited spatially separated expression of each antigen; CD31 was specifically associated with cells forming the vessel lumen, whereas CD140b was displayed on the surface of cells lining the exterior surface of the vessels. Taken together, these data demonstrate that ASCs are pericytic cells that occupy a perivascular position in vivo.

### Example 6

### ASCs Coassemble with and Stabilize Newly Forming EC Networks

To test for a functional correlate of the ASC-EC proximity in vascular structures, the effect of ASCs on formation and stabilization of EC network on Matrigel was evaluated. HMVECs cultured in growth factor-free media form temporary networks that started dissociating after 24 hours. Plating ASCs alone also revealed formation of networks on Matrigel. However, coculturing HMVECs with ASCs produced stabilized cell networks that remained intact for up to 5 days (n=9), when experiments terminated. Coculture of fluorescently labeled HMVECs and ASCs revealed that the cells formed a cooperative network of tubular structures on Matrigel matrix with HMVECs forming the lumen and ASCs overlaying tubes formed by HMVECs. Extending this finding into an in vivo system, subcutaneous implantation of collagen/fibronectin gels containing ASCs admixed with ECs into nude mice (n=4) also demonstrated formation of human-derived vascular structures with both endothelial and mural layers, with the mural layer comprising ASCs.

ASCs are components of the vascular wall functioning in paracrine support of microvasculature. The location of the ASCs in the vessel at the interface between endothelium and adipocytes and their ability to both support vascular structure and generate adipocytes expands the fact that they play an important role linking adipose tissue parenchymal mass with provision of its vascular supply.

Exogenous ASCs enhance vascular supply and limit ischemic tissue loss in models of limb ischemia.

### Example 7

### Paracrine Crosstalk Between ASCs and EC

The complementary effects of ASC-secreted factors on EC were evaluated. Unlike the finding with EC CM, ASC CM did not stimulate proliferation to cell numbers above those originally seeded but rather demonstrated a strong prosurvival effect on microvascular EC, markedly limiting their death in basal medium. HMVECs cultured in EBM-2/5% FBS for 4 days exhibit cell loss, with 50% remaining viable (P<0.001), whereas exposing cells to ASC CM (at 1:1 dilution) for the same time supports 100% cell survival (P<0.001). Evaluation for HMVEC chemotaxis toward ASC CM showed a modest but significant increase in migration compared with control media.

To evaluate factors responsible for such effects, proteins secreted by freshly isolated ASCs and cultured HMVECs were analyzed using antibody Arrays (RayBiotech). The protein profile in CM from ASCs revealed multiple angiogenic factors, including angiogenin, vascular endothelial growth factor, hepatocyte growth factor, bFGF, and β-nerve growth factor; the cytokines interleukin-6, -8, -11, and -17; and the cell-mobilizing factors monocyte chemoattractant proteins 1 and 2, granulocyte/macrophage colony-stimulating factor, and macrophage colony-stimulating factor. Notably absent were EGF, PDGF-BB, transforming growth factors, stromal cell-derived factor-1, and stem cell factor.

On the other hand, cultured HMVEC CM included multiple factors, such as bFGF, EGF, and PDGF-AA, -AB, and -BB. Several of these were quite distinct, whereas a few overlapped with the profile of ASC secretion (eg, bFGF). Quantitative analysis revealed that in HMVEC CM (72 hours), the concentration of factors were as follows: EGF, 83.6±51.0 μg/mL; bFGF, 243.0±46.4 μg/mL; and PDGF-BB, 405.2±85.0 μg/mL. The concentration of these in basal media before conditioning was below 2 pg/mL. The dose dependence of the effects of these factors on ASC proliferation was evaluated by the addition of each factor to basal medium. These factors exert strong mitogenic effects on ASCs over concentrations that overlap with those secreted by ECs. Further evaluation revealed synergy among factors with respect to mitogenesis of ASCs. Significantly higher mitogenesis of ASCs was observed to the factor mixture compared with individual factors at concentrations of 100 pg/mL for bFGF (P<0.05) and PDGF-BB (P<0.001) and 1 ng/mL for EGF (P<0.05) and PDGF-BB (P<0.01).

Exploration of the relationship of ASCs with ECs revealed their capacity for both structural and functional interactions. ASCs and EC in coculture exhibited preferential heterotypic assembly into vascular networks in vitro, which demonstrate a stability advantage in comparison with polygonal networks of ECs alone. This model for vascular assembly is suitable for in vitro screening and analyses of molecules mediating assembly by selective disruption of interactions. Exploration of paracrine interactions between ASCs and EC by CM transfer demonstrated mutual chemoattraction, consistent with their association in coculture. In addition, EC CM exhibited a mitogenic effect on ASCs, whereas ASC CM supported endothelial survival. Complementary factors secreted by HMVECs included isoforms of PDGF (AA, AB, and BB) as well as EGF, which showed potent mitogenic effects on ASCs, confirming functionality of PDGF receptors identified on ASCs by immunostaining. The heterotypic assembly as well as paracrine crosstalk are consistent with interactions described between microvascular ECs and pericytes from other sources.

### Example 8

### Effect of Serial vs. Simultaneous ECs and ASCs Plating on Vascular Network Formation

To evaluate whether morphogenesis of ECs to form vascular networks required their plating on the “feeder” ASC layer, experiments were conducted in which ECs and ASCs were mixed at a 1:12 ratio (EC:ASC, same ration as in serial plating method) and plated simultaneously. Analysis of VNF revealed that pre-formation of a stromal monolayer was not mandatory; in fact, following pre-mixture, ECs assembled into networks with higher efficiency (total tube lengths of 6.24±0.31 mm/mm2), compare with VNF on the monolayer, yielding total tube lengths of 4.87±0.47 mm/mm2 (p≦0.01).

Co-culture of EC and ASC: ASCs were plated on culture plastic at a density of 60×10³ cells/cm² in EBM-2/5% FBS media. In most experiments, ASCs were allowed to attach to the plates for three hours and then ECs from the indicated sources were plated on the top of ASC monolayer at densities ranging from 625 to 10,000 cells/cm². Cells were cultured in EBM-2/5% FBS for six days with media change at day 3. In a separate set of experiments, ASCs at 60×10³ cells/cm² and CBD-ECs at 5×10³ cells/cm² were premixed in EBM-2/5% FBS prior to plating.

In experiments assessing the role of various factors on EC-ASC interactions, co-cultures were incubated for 6 days in EBM-2/5% FBS media supplemented with: (1) neutralizing IgGs to: VEGF (RnD Systems, 2 μg/ml), HGF (RnD Systems, 2 μg/ml) or corresponding control mouse and goat IgGs (RnD Systems); (2) 10 μM GM6001 (Millipore, Billerica, Mass.), an inhibitor of MMP activity with a wide range of activity; (3) 50 μM AG1296 (Alexis, San Diego, Calif.), an inhibitor of PDGF receptor β activity; (4) and DMSO, which was used to dilute GM6001 and AG1296, as a negative control. Test cultures were exposed to the varying media conditions for six days.

Co-culture of ECs on ASC, NHDF and smooth muscle cell monolayers: Either ASCs, NHDFs, CASMCs, or AoSMCs were plated at a density of 60×10³ cells/cm² in EBM-2/5% FBS media, three hours prior to overlaying them with 5×10³ cells/cm² of CBD-ECs as previously described. The mixed cultures were incubated for six days with a routine media change at day 3. In experiments evaluating the effect of ASC conditioned media (ASC-CM) or added growth factors on vascular network formation, cells were cultured in EBM-2/5% FBS media that was either premixed with ASC-CM at a 1:1 ratio or supplemented with VEGF or HGF (10 ng/ml) as single growth factor controls. To generate ASC-CM, ASCs were grown to confluence in EGM-2mv in T75 flasks, followed by media replacement with EBM-2/5% FBS. After one day, 10 ml fresh EBM-2/5% FBS media was applied and removed after 72 hours conditioning. ASC-CM was collected, centrifuged at 300 g for five minutes, and supernatants frozen at 80° C. Cell number at the time of media collection was determined using hemocytometer.

In experiments evaluating the dependence of vascular network formation on ASC density, ASCs were premixed with NHDFs at ratios of 1:4, 1:9, 1:19 (ASC:NHDF). These cell mixtures were plated on cell culture plastic at densities of 60×10³ cells/cm². Three hours later CBD-ECs were seeded on top of the ASC-NHDF monolayers and incubated in EBM-2/5% FBS for six days.

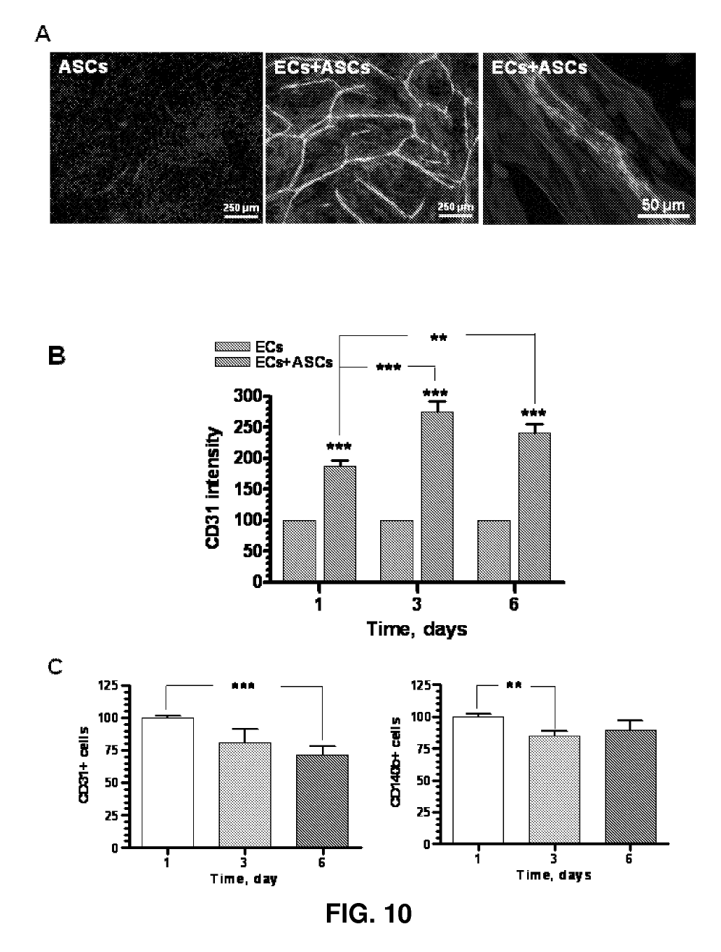

Culturing ASCs alone in EBM-2/5% FBS media resulted in relatively low and diffused expression of αSMA with a non-filamentous diffuse distribution (FIG. 10A). However, co-culturing of ASCs with ECs resulted both in significant increase in αSMA expression, and its organization into fibers, selectively in those ASCs which were in direct contact or close proximity with ECs (FIG. 10A). Although up-regulation of αSMA expression by ASCs was observed in EC-ASC coculture settings, expression of several other smooth muscle-specific antigens, such as desmin, caldesmon and calponin was not detected (not shown). Co-culturing also led to modulation of EC antigen expression, with an increase of extracellular presentation of PECAM/CD31 by up to 275±16% at day 3 in comparison with EC culture alone (p<0.001) (FIG. 10B). There was no further change in level of CD31 expression between day 3 and day 6 (length of experiment).

### Example 9

### Extracellular Matrix Protein Production by ASCs in EC-ASC Co-Culture System

Exposure of ASCs to ECs resulted in a significant increase in laminin expression compared to the low level of expression observed in ASC mono-cultures by immunofluorescence. Double-staining of the wells with anti-CD31 and laminin IgGs revealed that the pattern of intense laminin staining mirrored the geometry of EC cord structures. The laminin, produced by ASCs, coated the outer surfaces of the EC network. Evident fibronectin expression was noted in ASC monocultures; however, the staining mostly exhibited intracellular localization. In contrast, co-culture with ECs redistributed fibronectin localization into the extracellular space and resulted in its organization into fibrils. Perlecan I expression and distribution was not detected in ASC monoculture, whereas in co-culture the perlecan I expression was observed.

This study demonstrated that exposure of ASCs to ECs increased the levels of laminin and fibronectin expression compared to ASCs monocultures. Expression of perlecan-1 was only detectable in co-cultures. This indicates that accumulation of ECM proteins in the areas of ASC localization adjacent to EC cords produces favorable conditions (including creating binding sites for integrins and growth factors) for ECs to assemble into premature vessels. This hypothesis is supported by observations that ECs are able to spontaneously assemble into cord structures on Matrigel, whose primary protein is laminin (60%). The hypothesis that ECM protein play a crucial role in VNF is further supported by observation that blockage of MMP activity led to significant reduction of VNF. MMPs are responsible for ECM protein remodeling and growth factor activation, and the finding that their inhibition limits network formation supports the theory that ASC-produced ECM augments VNF.

### Example 10

### Effect of Neutralizing IgGs and Chemicals on Vascular Network Formation

To evaluate the importance of several angiogenic factors for VNF, co-cultures were exposed to basal media containing neutralizing IgGs to VEGF or HGF, factors secreted in significant quantities by ASCs. In the presence of anti-VEGF IgGs in the media, total tube lengths decreased from 5.75±0.4 mm/mm² (control, in the presence of mIgG_{2b}) to 2.23±0.4 mm/mm² (p≦0.01). The degree of network branching decreased to a proportionally greater degree: from 10.18±1.36 branches/mm² for mIgG_{2b} to 2.18±0.68 branches/mm² for anti-VEGF IgGs (p≦0.01). Interestingly anti-HGF treatment inhibited total tube length in the networks: 4.01±0.22 mm/mm² for anti-HGF IgGs vs. 5.58±0.24 mm/mm² for mIgG₁ (p≦0.01), but had proportionally less effect on degree of network branching: 9.36±0.78 branches/mm² with anti-HGF IgGs vs. 10.96±0.97 branches/ mm² with mIgG₁.

The role of PDGF-BB, which is secreted in significant amount by ECs (not shown), in VNF was evaluated by exposing cells to the chemical inhibitor of PDGF-BB receptor—AG1296 (50 μM). PDGF-BB inhibition led to marked reduction of VNF in total tube length: 1.07±0.14 mm/mm² for AD1296 vs. 5.29±0.31 mm/mm² for DMSO (p≦0.001). The PDGF-BB inhibition also reduced the degree of network branching: 0.26±0.06 branches/mm² for AG1296 vs. 5.88±0.67 branches/mm² for DMSO (p≦0.001).

The role of MMPs in VNF was tested by exposing co-cultures to the media containing 10 μM of GM6001, an inhibitor of MMP-1, 2, 3, 8, 9, or to DMSO as a control. Based on total tubes length, 2.59±0.05 mm/mm² for GM6001 vs. 4.24±0.31 mm/mm² for control (p≦0.01), and the degree of network branching, 2.45±0.17 per mm² for GM6001 vs. 5.86±0.79 per mm² for control (p≦0.01), it was concluded that MMPs also played an essential role in VNF both with respect to overall assembly as well as branch formation.

The experiments with neutralizing antibodies to angiogenic factors support in vivo observations that VEGF and HGF, factors that are secreted by ASCs in significant amounts, as well as PDGF-BB, of endothelial origin, are powerful positive modulators of EC-ASC VNF. Blocking the activities of VEGF and HGF in the EC-ASC cultures significantly blocked the VNF. Conversely, exogenous introduction of these factors to EC-NHDF and EC-SMCs co-cultures significantly promoted VNF. However, neither VEGF, nor HGF, nor ASC-CM could be used to replace the effects of ASC presence in co-cultures on VNF. Therefore, direct interaction between EC and ASC may play a role in efficient VNF.

### Example 11

### Comparative Analysis ASCs, SMCs and Fibroblasts in Potential to Support VNF

To evaluate whether the ability to stimulate VNF by ECs is a unique characteristic of ASCs or if other cell types possess similar activity, comparative analysis of VNF by ECs were performed on monolayers of ASCs, smooth muscle cells (SMCs) and fibroblasts. Anti-CD31 staining revealed that ASCs promote VNF much strongly than the other tested cell types based on total tube length (3.69±0.19 mm/mm²) and network branching (5.58±0.7 per mm²). SMCs and fibroblasts promoted weakly formed networks with total tube length 0.92±0.06 mm/mm² for CASMCs, 1.65±0.08 mm/mm² for AoSMCs, and 1.14±0.23 mm/mm² for fibroblasts (p≦0.001 ASC vs. all other cell types) and network branching: 0.91±0.18 per mm² for CASMCs, 2.11±0.24 per mm² for AoSMCs, and 1.06±0.45 per mm² for fibroblasts (p≦0.001 ASCs vs. CASMCs and fibroblasts, p≦0.01 ASCs vs AoSMCs).

To evaluate the role of the soluble factors secreted by ASCs, co-cultures of ECs with ASCs, SMCs and fibroblasts were exposed for 6 days either to ASC-CM or to VEGF and HGF, both factors secreted by ASCs. In EC-ASC co-cultures, only VEGF supplementation significantly augmented network development, increasing total tube length by 76%. EC co-cultured on CASMC or fibroblast monolayers in the presence of ASC-CM resulted in increased total tube lengths by 3.12 and 4.36 times, respectively, by comparison with co-culture in control media (EC+CASMC control: 0.37±0.13 mm/mm²; EC+CASMC+ASC-CM: 1.12±0.08 mm/mm²; EC+fibroblast control: 0.54±0.1 mm/mm²; EC+fibroblast+ASC-CM: 2.43±0.07 mm/mm²). A similar trend was observed when co-cultures were exposed to VEGF (EC+CASMC+VEGF: 0.9±0.18 mm/mm²; EC_fibroblast+VEGF: 3.2±0.4 mm/mm²) and HGF (EC+CASMC+HGF: 0.74±0.21 mm/mm²; EC+fibroblasts+HGF: 1.0±0.02 mm/mm². While supplementation by ASC-CM,VEGF, or HGF all had significant potentiation of VNF with SMCs and fibroblastsVNF, none of these treatment was able to reach the degree of VNF demonstrated by direct contact with ASC in control media.

To evaluate whether VNF stimulation was dependent on preconditioning “feeder” cells on the certain media, ASCs and fibroblasts were expanded for 2 passages either in their typical media (ASC with EGM2-MV and fibroblasts with FGM-2), or in the media normally used for expanding the alternative cell type: ASCs in FGM-2 and fibroblasts in EGM-2mv. The ASCs and fibroblasts were subsequently co-cultured with CBD-ECs. Analysis of the networks at day 6 revealed that the potential of the ASC to stimulate EC VNF was not affected by the media used for ASC expansion. Fibroblasts expanded in EGM-2mv media had slight stimulatory effects on VNF, but this effect was approximately threefold lower than the network development observed with ASCs expanded in either FGM-2 or EGM-2mv medium.

To evaluate whether the physical presence of ASCs would stimulate VNF in an EC−fibroblast system, ASC were premixed with NHDF in ratios from 1:19-1:4 (ASC:NHDF) prior to plating, followed by overlay with ECs. Comparative analysis of the network formed by ASCs and fibroblasts alone or in their mixtures demonstrated that the presence of ASCs in fibroblasts monolayer were able to dose-dependently increase total tube length of the vascular network. To evaluate if this effect was exclusively due to ASC paracrine action or also occurred in the context of direct interaction of ASCs with ECs, ASCs were labeled with the fluorescent dye DiI prior to mixing with fibroblasts. Analysis of co-localization of ECs (stained for CD31 antigen) with DiI-labeled ASCs at day 6 revealed that ASCs were localized in close proximity to EC cords. Additionally, the relative density of labeled ASC was markedly depleted in areas distant from the EC cords, demonstrating clear tropism of ASC towards endothelial tubes, even the context of an excess of fibroblasts. This phenomenon helps explain the observation that VNF is associated with increases nuclear density (of ASC) in regions proximate to the EC cord structures.

The present in vitro study revealed that ASCs that were adjacent to ECs specifically displayed induction of αSMA and its organization into fibers, suggesting the hypothesis that ECs induce a differentiation program in ASCs including αSMA expression and fibrillogenesis, as well as laminin, fibronectin, and perlecan expression (FIG. 10). However, increase in αSMA expression was not associated with increased expression of several other smooth muscle cell antigens, including caldesmon, desmin and calponin, indicating that in this model ASCs do not undergo differentiation to smooth muscle cell.

The results of this study strongly indicate that ASCs exhibit a significantly higher potential to promote vascular network formation in concert with ECs than either SMCs or fibroblasts. Despite that ASCs conditioned media or angiogenic factors, such as VEGF or HGF stimulate VNF on SMCs and fibroblasts, none of these supplements were able to substitute for contact with ASCs in the co-culture system. Further, soluble factors secreted by ASCs were not sufficient to promote efficient VNF, which indicate that physical interaction between ECs and ASCs is crucial for this process, that ASCs are induced to secrete a more potent offset of vasculogenic factors only when they are in direct contact/interaction with ECs, or perhaps both. These hypotheses are well supported by the observation that even low ASC doses (5-20%) significantly promote VNF.

1. A method of evaluating vasculogenic potential of endothelial cells, the method comprising:
(a) culturing the endothelial cells (EC) and a plurality of adipose stromal cells (ASC); and
(b) evaluating the vasculogenic potential by the formation of a vascular network.

2. The method of claim 1, wherein the endothelial cells are cultured on a layer of adipose stromal cells.

3. The method of claim 1, wherein the endothelial cells and adipose stromal cells are pre-mixed prior to culturing.

4. The method of claim 1, wherein the endothelial cells are derived from a source selected from the group consisting of placenta, peripheral blood, vessel wall, cord blood, umbilical vein, cardiac microvascular cells, adipose tissue, saphenius vein, HUVEC, HmVEC, and ECFC.

5. The method of claim 1, wherein the adipose stromal cells are adherent.

6. The method of claim 1, wherein the adipose stromal cells are CD34⁺CD45−CD31⁻.

7. The method of claim 5, wherein the adherent cells express a marker selected from the group consisting of CD10, CD13, and CD90.

8. The method of claim 1, wherein the adipose stromal cells express a marker selected from the group consisting of chondroitin sulfate proteoglycan (NG2), CD140a, and CD140b (PDGF receptor- and -β, respectively).

9. The method of claim 1, wherein the endothelial cells are selected from the group consisting of mature endothelial cells, endothelial progenitor cells (EPC) and endothelial colony forming cells (ECFC).

10. The method of claim 1, wherein the endothelial cells are CD31⁺CD144⁺.

11. The method of claim 2, wherein the wherein the adipose stromal cells are grown as a monolayer.

12. The method of claim 1, wherein the adipose stromal cells are seeded at an initial density of about 3,500-240,000 cells/cm² and the endothelial cells are seeded at an initial density of about 625-25,000 cells/cm².

13. The method of claim 1, wherein the adipose stromal cells and endothelial cells are mixed at a ratio of about 5:1 to about 100:1.

14. The method of claim 1, wherein the adipose stromal cells are cultured for about 3 hours prior to adding the endothelial cells.

15. The method of claim 1, wherein the adipose stromal cells and the endothelial cells are generally homogenous and substantially pure.

16. The method of claim 1, wherein the adipose stromal cells and the endothelial cells are cultured substantially free of one or more exogenously introduced components of the extracellular matrix (ECM).

17. A method of screening a candidate angiogenic or anti-angiogenic agent, the method comprising:
(a) culturing adipose stromal cells and endothelial cells capable of forming a vascular network in vitro;
(b) contacting adipose stromal cells and endothelial cells with the candidate agent; and
(c) determining that the candidate agent is angiogenic or anti-angiogenic if the candidate compound augments angiogenesis or inhibits angiogenesis by the comparing the vascular network formation to a control sample without the candidate compound.

18. An assay kit comprising an effective amount of endothelial cells and adipose stromal cells (ASC), wherein the adipose stromal cells and endothelial cells (EC) are mixed at a ratio of about 5:1 to about 100:1.

19. The kit of claim 29, wherein the cells are frozen in a vial.

20. The kit of claim 29, wherein the ASC are about 200,000-5,000,000 cells and the endothelial cells are about 200,000-2,000,000 cells.
